(19) United States
(12) Patent Application Publication
(10) Pub. No.: US 2021/0353387 A1
(43) Pub. Date: Nov. 18, 2021

(54) DENTAL APPLIANCE WITH STRUCTURED SURFACE

(71) Applicant: 3M INNOVATIVE PROPERTIES COMPANY, St. Paul, MN (US)

(72) Inventors: Bhaskar V. Velamakanni, Woodbury, MN (US); Paul R. Klaiber, Mahtomedi, MN (US); Richard P. Rusin, Woodbury, MN (US); Daniel J. Skamser, Ham Lake, MN (US); Negus B. Adefris, St. Paul, MN (US)

(21) Appl. No.: 17/284,540

(22) PCT Filed: Oct. 11, 2019
(86) PCT No.: PCT/IB2019/058712

§ 371 Date: Apr. 12, 2021

Related U.S. Application Data
(60) Provisional application No. 62/747,264, filed on Oct. 18, 2018.

(51) Int. Cl. A61C 7/08; A61C 19/06; A61K 6/69

(52) U.S. Cl. CPC A61C 7/08; B29C 51/14; A61K 6/69; A61C 19/063

(57) ABSTRACT

A dental appliance is described, the dental appliance including a polymeric substrate with a plurality of cavities for receiving one or more teeth, an arrangement of engineered microstructures on the substrate wherein the engineered microstructures include a therapeutic agent. The microstructures may be three-dimensionally engineered on a polymeric film disposed on a major surface of the polymeric substrate, wherein the microstructures extend outwards from the surface. The microstructures also comprise a compound releasable from the three-dimensionally engineered microstructures over a predetermined patient wear time.

### BACKGROUND

Orthodontic treatments involve repositioning misaligned teeth and improving bite configurations for improved cosmetic appearance and dental function. Repositioning teeth is accomplished by applying controlled forces to the teeth over an extended time period.

In one example, teeth may be repositioned by placing a dental appliance, generally referred to as an orthodontic aligner or an orthodontic aligner tray, over the teeth of the patient for each treatment stage of an orthodontic treatment. The orthodontic alignment tray includes a polymeric shell defining a plurality of cavities for receiving one or more teeth. The individual cavities in the polymeric shell are shaped to exert force on one or more teeth to resiliently and incrementally reposition selected teeth or groups of teeth in the upper or lower jaw.

A series of orthodontic aligner trays are provided to a patient to be worn sequentially and alternatingly during each stage of the orthodontic treatment to gradually reposition teeth from one tooth arrangement to a successive tooth arrangement to achieve a desired tooth alignment condition. Once the desired alignment condition is achieved, an aligner tray, or a series of aligner trays, may be used periodically or continuously in the mouth of the patient to maintain tooth alignment. In addition, orthodontic retainer trays may be used for an extended time period to maintain tooth alignment following the initial orthodontic treatment.

In some examples, a stage of orthodontic treatment may require that an aligner tray remain in the mouth of the patient for several hours a day, over an extended time period of days, weeks or even months. While the orthodontic aligner tray is in use in the mouth of the patient, foods or other substances can stain or otherwise damage the appliance. In addition, microorganisms can contaminate the surface of the appliance, which in some cases can also cause biofilms to form on the surface. The biofilms can be difficult to remove, even if the appliance is periodically cleaned. Microorganisms or biofilm buildup on the surface of the aligner tray can stain or otherwise discolor the aligner tray, can cause undesirable tastes and odors, and even potentially lead to various periodontal diseases.

In some examples, thermoplastic aligner trays can expand from viscoelastic creep/stretch and hydration-induced plasticization, typically within one-week of being in a patient's mouth. This undesirable stretching reduces the effectiveness of the aligner tray, and in some cases can result in patient discomfort.

### SUMMARY

In general, the present disclosure is directed to a dental appliance that includes a polymeric substrate with a plurality of cavities for receiving one or more teeth. The polymeric substrate includes an arrangement of engineered microstructures on the substrate, wherein the engineered microstructures include a therapeutic agent.

To help reduce or even prevent damage to a dental appliance and the teeth of a patient caused by biofilm buildup, in one aspect the present disclosure is directed to a dental appliance that includes a three-dimensional (3D) textured surface. In some embodiments, the individual structures on the 3D textured surface are configured to release therapeutic agents in a patient's mouth to, for example, protect the teeth against decalcification, and/or to prevent biofilm formation on the exposed surfaces of the dental appliance.

In some embodiments, the dental appliance includes a polymeric substrate with a plurality of cavities for receiving one or more teeth and a polymeric film on a major surface of the polymeric substrate. In some embodiments, an outwardly facing surface of the polymeric film includes an array of three-dimensional engineered microstructures extending upward from the outwardly facing surface, and the three-dimensional engineered microstructures include a compound releasable from the three-dimensional engineered microstructures over a predetermined patient wear time of the dental appliance.

In some embodiments, the dental appliance includes a polymeric substrate with a plurality of cavities for receiving one or more teeth and an arrangement of engineered microstructures on the substrate. In some embodiments, the engineered microstructures include a therapeutic agent.

The present disclosure is further generally directed to a method of making a dental appliance. The method may include attaching to a first major surface of a first polymeric film a first major surface of a second polymeric film to form a laminate construction, wherein a second major surface of the second polymeric film comprises an array of three-dimensional engineered microstructures extending outward therefrom, and wherein the three-dimensional engineered microstructures comprise a compound releasable from the three-dimensional engineered microstructures. The method may also include shaping the laminate construction to comprise an arrangement of cavities configured to receive one or more teeth.

In some examples, the present disclosure describes another method of making a dental appliance. The method may include casting a liquid polymeric resin on a tool comprising a plurality of engineered microstructures to form a microstructured film, wherein the microstructured film comprises a first major surface and a second major surface. The method may also include applying a polymeric film on the first major surface of the microstructured film. The method may additionally include removing the tool to form a laminate construction, wherein the second major surface of the microstructured film comprises an array of three-dimensional engineered microstructures extending outward therefrom, and wherein the three-dimensional engineered microstructures comprise a compound releasable from the three-dimensional engineered microstructures. The method may also include forming in the laminate construction a plurality of cavities configured to accept one or more teeth and create the dental appliance.

In some examples, the present disclosure describes an additional method of making a dental appliance. The method may include applying a layer of an adhesive to a major surface of a polymeric film and hardening the adhesive to form a hardened adhesive layer. The method may also include casting a liquid polymeric resin on a tool comprising a plurality of engineered microstructures to form a microstructured film, wherein the microstructured film comprises a first major surface and a second major surface. The method may additionally include laminating the polymeric film to the microstructured film, wherein the adhesive layer is on the first major surface of the microstructured film. The method may also include hardening the liquid polymeric resin and removing the tool to form a laminate construction, wherein the second major surface of the microstructured film comprises an array of three-dimensional engineered microstructures extending outward therefrom, and wherein the three-dimensional engineered microstructures comprise a compound releasable from the three-dimensional engineered microstructures. The method may further include forming in the laminate construction a plurality of cavities configured to accept one or more teeth and create the dental appliance.

In another aspect, the present disclosure is directed to a method of dental treatment. The method may include positioning a dental appliance around one or more teeth, wherein the dental appliance comprises a plurality of cavities for receiving one or more teeth, and an array of three-dimensional engineered microstructures on an exposed surface of the dental appliance, wherein the engineered microstructures comprise one or more therapeutic agents. The method may also include releasing the therapeutic agents into the mouth of a patient.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic overhead perspective view of an example orthodontic appliance.

FIGS. 2A-2C are schematic top views of example three-dimensional micro textured surfaces that can be used to form part of the orthodontic appliance of FIG. 1.

FIG. 3 is a schematic overhead perspective view of a method for using an orthodontic appliance by placing the orthodontic appliance to overlie teeth.

FIG. 4 is a schematic of a cross-sectional view of an example orthodontic appliance.

FIG. 5 is a schematic of a cross-sectional view of example subassembly of orthodontic appliance components for an orthodontic appliance.

FIGS. 6A and 6B are cross-sectional and top views, respectively, of an example microreplication tool.

FIG. 7 is a schematic of a cross-sectional view of an example subassembly for an orthodontic appliance.

FIG. 8 is a schematic of a cross-sectional view of an example subassembly for an orthodontic appliance.

FIG. 9 is a schematic of a cross-sectional view of an example preliminary orthodontic appliance.

FIG. 10 is a schematic of a cross-sectional view of an example orthodontic appliance.

FIG. 11 is a topographical view of a component of an example preliminary orthodontic appliance.

Like symbols in the drawings indicate like elements.

### DETAILED DESCRIPTION

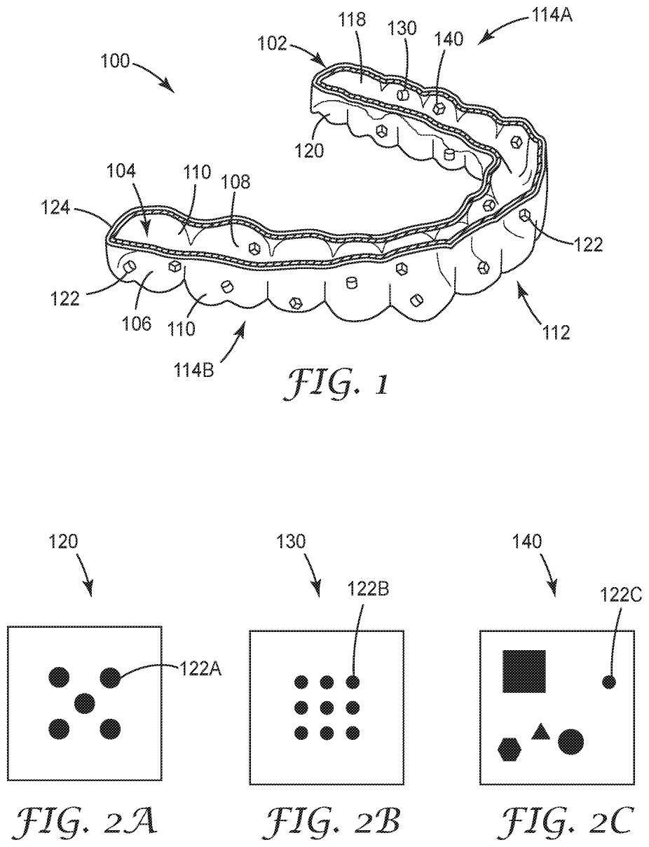

FIG. 1 is a schematic overhead perspective view of a dental appliance 100, which is also referred to herein as an orthodontic aligner tray or retainer tray. The dental appliance 100 includes a polymeric shell 102 with a plurality of cavities 104. The cavities 104 are shaped to receive and resiliently reposition one or more teeth in an upper or lower jaw of a patient from one tooth arrangement to a successive tooth arrangement, or to receive and maintain the position of the previously realigned one or more teeth. The polymeric shell 102 includes a first major external surface 106 and a second major internal surface 108 that contacts the teeth of a patient, at least one of which includes a layer 124 with a 3D micro structured surface 110 with an arrangement of microstructures 122 extending outward therefrom. The polymeric shell 102 further includes a front 112, a left side 114A and a right side 114B. In FIG. 1, the polymeric shell 102 is shown as being shaped for an upper arch. The polymeric shell 102 can also be for a lower arch.

The shell 102 of the orthodontic appliance 100 is an elastic polymeric material that generally conforms to a patient's teeth, and may be transparent, translucent, or opaque. In some embodiments, the shell 102 is a clear or substantially transparent polymeric material that may include, for example, one or more of amorphous thermoplastic polymers, semi-crystalline thermoplastic polymers and transparent thermoplastic polymers chosen from polycarbonate, thermoplastic polyurethane, acrylic, polysulfone, polypropylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone and combinations thereof. In another embodiment, the shell 102 may be chosen from clear or substantially transparent semi-crystalline thermoplastic, crystalline thermoplastics and composites, such as polyamide, polyethylene terephthalate. polybutylene terephthalate, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof. In some embodiments, the shell 102 is a polymeric material chosen from polyethylene terephthalate, polyethylene terephthalate glycol, polycyclohexylenedimethylene terephthalate glycol, and mixtures and combinations thereof. One example of a commercially available material suitable as the elastic polymeric material for the shell 102, which is not intended to be limiting, is polyethylene terephthalate (polyester with glycol additive (PETg)). Suitable PETg resins can be obtained from various commercial suppliers such as, for example, Eastman Chemical, Kingsport, Tenn.; SK Chemicals, Irvine, Calif.; DowDuPont, Midland, Mich.; Pacur, Oshkosh, Wis.; and Scheu Dental Tech, Iserlohn, Germany.

In some embodiments, the shell 102 may be made of a single polymeric material or may include multiple layers of different polymeric materials.

In one embodiment, the shell 102 is a substantially transparent polymeric material. In this application, the term substantially transparent refers to materials that pass light in the wavelength region sensitive to the human eye (about 0.4 micrometers (μm) to about 0.75 μm) while rejecting light in other regions of the electromagnetic spectrum. In some embodiments, the reflective edge of the polymeric material selected for the shell 102 should be above about 0.75 μm, just out of the sensitivity of the human eye.

In various embodiments, the shell 102 has a thickness of less than 1 mm, but varying thicknesses may be used depending on the application of the orthodontic appliance 100. In various embodiments, the shell 102 has a thickness of about 50 μm to about 3,000 μm, or about 300 μm to about 2,000 μm, or about 500 μm to about 1,000 μm, or about 600 μm to about 700 μm.

In some embodiments, the layer 124 including the structured surface 110 is substantially transparent to visible light of about 400 nm to about 750 nm when applied at a thickness of about 50 μm to about 1000 μm on a substantially transparent polymeric shell 102. In various embodiments, the visible light transmission through the combined thickness of the shell 102 and the layer 124 is at least about 50%, or about 75%, or about 85%, or about 90%, or about 95%.

In one embodiment, the microstructures 122 on the structured surface 110 are configured to release one or more therapeutic agents that can have a beneficial effect in the mouth of the patient. Examples include, but are not limited to, fluoride sources, whitening agents, anticaries agents (e.g., xylitol), re-mineralizing agents (e.g., calcium phosphate compounds), enzymes, breath fresheners, anesthetics, clotting agents, acid neutralizers and pH control agents, ion-recharging agents, chemotherapeutic agents, immune response modifiers, thixotropes, polyols, anti-inflammatory agents, antimicrobial agents, antifungal agents, agents for treating xerostomia, desensitizers, and the like, of the type often used in dental compositions. Combination of any of the above therapeutic agents may be used.

In some embodiments, suitable therapeutic agents include re-mineralizing agents such as calcium, phosphorous, and fluoride compounds.

For example, in some embodiments, suitable calcium compounds include, but are not limited to, calcium chloride, calcium carbonate, calcium caseinate, calcium chloride, calcium citrate, calcium glubionate, calcium gluceptate, calcium glycerophosphate, calcium gluconate, calcium hydroxide, calcium hydroxyapatite, calcium lactate, calcium oxalate, calcium oxide, calcium pantothenate, calcium phosphate, calcium polycarbophil, calcium propionate, calcium pyrophosphate, calcium sulfate, and mixtures and combinations thereof. These compounds have been found to minimize demineralization of calcium hydroxyapatite at the surface of the tooth of a patient.

In some embodiments, the tooth re-mineralizing compounds include phosphate compounds. Suitable phosphate compounds include, but are not limited to, aluminum phosphate, bone phosphate, calcium phosphate, calcium orthophosphate, calcium phosphate dibasic anhydrous, calcium phosphate-bone ash, calcium phosphate dibasic dihydrate, calcium phosphate dibasic anhydrous, calcium phosphate dibasic dihydrate, calcium phosphate tribasic, dibasic calcium phosphate dihydrate, dicalcium phosphate, neutral calcium phosphate, precipitated calcium phosphate, tertiary calcium phosphate, tricalcium phosphate, whitlockite, magnesium phosphate, potassium phosphate, dibasic potassium phosphate, dipotassium hydrogen orthophosphate, dipotassium monophosphate, dipotassium phosphate, monobasic potassium phosphate, potassium acid phosphate, potassium biphosphate, potassium dihydrogen orthophosphate, potassium hydrogen phosphate, sodium phosphate, anhydrous sodium phosphate, dibasic sodium phosphate, disodium hydrogen orthophosphate, disodium hydrogen orthophosphate dodecahydrate, disodium hydrogen phosphate, disodium phosphate, and sodium orthophosphate.

Fluoride compounds incorporated into the mineral surface of a tooth help inhibit the demineralization of enamel and protect the tooth. Fluoride compounds absorbed into mineral surfaces of a tooth attract calcium and phosphate ions from saliva, or other sources, which results in the formation of fluorapatite and protects the tooth against demineralization. While not wishing to be bound by any theory, currently available evidence indicates that fluorapatite exhibits lower solubility than naturally occurring hydroxyapatite, which can help resist the inevitable acid challenge that teeth face daily.

Orthodontic patients are considered high risk for caries over the course of their treatment. Commercial fluoride varnishes are very sticky by design and typically last a few hours on the enamel once applied. For an orthodontic patient wearing a dental appliance such as an aligner tray, this is undesirable since the varnish can interfere with the fit of the aligners on the arches of the patient, as well as adhere to the plastic that the aligners are made from and permanently warp or deform them. In one embodiment, for example, the microstructures 122 on the structured surface 110 can be configured to deliver beneficial fluoride over a typical wear time for an alignment tray set (for example, 7 days), without compromising the fit of the alignment tray for the patient or ruining the polymeric material from which the alignment tray is made.

In some embodiments, the calcium compounds, phosphate compounds, fluoride compounds or combinations thereof, are present in the microstructures 122 on the structured surface 110 in an amount sufficient such at least one of calcium, phosphate or fluoride can substantially reduce or prevent demineralization on the surface of the teeth of the patient that are adjacent to or contacting the internal surface 108 of the polymeric shell 102 during or exceeding a predetermined wear time.

In another embodiment, the therapeutic agents in the microstructures 122 include compounds selected to reduce the bacteria on at least one of the internal surface 108 and the external surface 106 of the polymeric shell 102. Suitable antibacterial or biofilm-reducing compounds include, but are not limited to, biocompatible metal oxides such as, silver oxide, copper oxide, gold oxide, zinc oxide, magnesium oxide, titanium oxide, chromium oxide, and mixtures, alloys and combinations thereof.

The microstructures 122 can include any antimicrobially effective amount of metal oxide MOx. In various embodiments, which are not intended to be limiting, the microstructures 122 can include less than 100 mg, less than 40 mg, less than 20 mg, or less than 5 mg MOx per 100 cm². The metal oxide can include, but is not limited to, silver oxide, copper oxide, gold oxide, zinc oxide, magnesium oxide, titanium oxide, chromium oxide, and mixtures, alloys and combinations thereof. In some embodiments, which are not intended to be limiting, the metal oxide can be chosen from AgCuZnOx, Ag doped ZnOx, Ag doped AZO, Ag doped TiO2, Al doped ZnO, and TiOx.

In some embodiments, the microstructures 122 can include one or more antibacterial agents. Examples of suitable antibacterial agents can include, but are not limited to, aldehydes (glutaraldyde, phthalaldehyde), salts of phenolics or acids, chlorhexidine or its derivatives (including acid adducts such as acetates, gluconates, chlorides, nitrates, sulfates or carbonates), and combinations thereof.

Non-limiting examples of antibacterial agents include: zinc salts, zinc oxide, tin salts, tin oxide, benzalkonium chloride, hexitidine, long chain alkyl ammonium or pyridinium salts (e.g., cetypyridinium chloride, tetradecylpyridinium chloride), essential oils (e.g., thymol), furanones, chlorhexidine and salt forms thereof (e.g., chlorhexidine gluconate), sanguinarine, triclosan, stannous chloride, stannous fluoride, octenidine, non-ionic or ionic surfactants (e.g., quaternary ammonium compounds), alcohols (monomeric, polymeric, mono-alcohols, poly-alcohols), aromatic alcohols (e.g., phenol)), antimicrobial peptides (e.g., histatins), bactericins (e.g., nisin), antibiotics (e.g., tetracycline), aldehydes (e.g., glutaraldehyde) inorganic and organic acids (e.g., benzoic acid, salicylic acid, fatty acids, etc.) or their salts, derivatives of such acids such as esters (e.g., p-hydroxybenzoates or other parabens, glycerol esters of fatty acids such as lauricidin), silver compounds, silver salts, silver nanoparticles, peroxides (e.g., hydrogen peroxide), and combinations thereof.

In various embodiments, the therapeutic agents released from the microstructures 122 can vary between regions on a surface of the polymeric shell 102 and even within a single region. For example, the therapeutic agents released by the microstructures 122 in a first region 120 of the polymeric shell 102 can be different from the therapeutic agents released from the microstructures 122 in a second region 130, e.g., fluoride in the first region 120 and phosphate in the second region 130. In other examples, the therapeutic agents released from the microstructures 122 within the first region 120 can differ from one another, e.g., fluoride and phosphate could be released from different microstructures 122 in the first region 120.

In another embodiment, the therapeutic agents within the microstructures 122 can be released at a different concentration between regions and within a selected region. For example, the therapeutic agents of the microstructures 122 in the first region 120 can be released at a different concentration than the therapeutic agents of the microstructures 122 in the second region 130. In some examples, therapeutic agents of the microstructures 122 in the first region 120 can be released at varying concentrations. In other examples, therapeutic agents can be released at the same concentrations across all regions or in different areas of the polymeric shell 102 such as, for example, along the front 112 or along the sides 114A, 114B.

In another embodiment, the therapeutic agents released from the microstructures 122 can be releasable over a predetermined patient wear time of the orthodontic appliance 100. In some examples, the therapeutic agents may be released over a period of seconds, minutes, hours, days, weeks, or months. In addition, different regions of the orthodontic appliance can have therapeutic agents with varying predetermined release periods. For example, one region may have a release period on the order of seconds, and another different region may have a release period on the order of months.

In another embodiment, the microstructures 122 on the structured surface 110 can absorb and release therapeutic agents. For example, calcium and/or phosphorus can be absorbed from saliva and released over time. In another example, fluoride, calcium, tin, and/or phosphorus can be absorbed from oral care products (e.g., toothpaste and rinse) and released over time.

In another embodiment, the microstructures 122 on the structured surface 110 can be made from an elastomeric polymeric material selected to, for example, ease placement and removal of the dental applicant in the mouth of the patient, improve comfort against the teeth or the tissues in the mouth of the patient, or enhance tray-to-dentition contact area leading to lower stress and/or effective force transfer from the structured surface 110 for repositioning teeth. In some examples, elastomers can include polyisoprenes, polybutadienes, chloroprene rubbers, butyl rubbers, halogenated butyl rubbers, fluoropolymers, nitriles, ethylene propylene rubbers, ethylene propylene diene rubbers, silicone rubbers, polyacrylic rubbers, fluorosilicones, fluoroelastomers, polyether block amides, cholorsulfinated polyethylenes, and ethylene vinyl acetates.

In another embodiment, the microstructures 122 on the structured surface 110 can facilitate unhindered flow of salivary fluids and other fluids to enhance and/or maintain the hard tissue health. When a tooth surface undergoes demineralization instigated by oral bacteria, dietary choices, xerostomia, etc., the microstructures 122 on the structured surface 110 can provide open channels for the saliva to re-mineralize and hydrate the tooth surface. The free flow of saliva can also enhance the release of therapeutic agents, improving the efficacy of treatment. The microstructures may also provide an increased surface area for release of therapeutic agent into the salivary flow, which can facilitate unhindered release of fluoride or other agents.

As shown in FIG. 1, in an embodiment the structured surface 110 can be formed as a polymeric film 124. In some embodiments, the polymeric film 124 is cast from a resin composition including a suitable resin matrix and a therapeutic compound incorporated into the resin matrix.

Suitable resins include, but are not limited to, epoxy resins (which contain cationically active epoxy groups), vinyl ether resins (which contain cationically active vinyl ether groups), ethylenically unsaturated compounds (which contain free radically active unsaturated groups, e.g., acrylates and methacrylates), and combinations thereof. Also suitable are polymerizable materials that contain both a cationically active functional group and a free radically active functional group in a single compound. Examples include epoxy-functional (meth)acrylates.

As used herein, ethylenically unsaturated compounds with acid functionality includes monomers, oligomers, and polymers having ethylenic unsaturation and acid and/or acid-precursor functionality. Acid-precursor functionalities include, for example, anhydrides, acid halides, and pyrophosphates. Ethylenically unsaturated compounds with acid functionality include, for example, α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl) phosphate, ((meth)acryloxypropyl) phosphate, bis((meth)acryloxypropyl) phosphate, bis((meth)acryloxy)propyloxy phosphate, (meth)acryloxyhexyl phosphate, bis((meth)acryloxyhexyl) phosphate, (meth)acryloxyoctyl phosphate, bis((meth)acryloxyoctyl) phosphate, (meth)acryloxydecyl phosphate, bis((meth)acryloxydecyl) phosphate, 10-methacryloyloxydecyl dihydrogen phosphate (MDP monomer), caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, poly(meth)acrylated polyboric acid, and the like, may be used as components in the hardenable resin system. Also, monomers, oligomers, and polymers of unsaturated carbonic acids such as (meth)acrylic acids, aromatic (meth)acrylated acids (e.g., methacrylated trimellitic acids), and anhydrides thereof can be used. Some compositions can include an ethylenically unsaturated compound with acid functionality having at least one P—OH moiety.

Photopolymerizable compositions may include compounds having free radically active functional groups that may include monomers, oligomers, and polymers having one or more ethylenically unsaturated group. Suitable compounds contain at least one ethylenically unsaturated bond and are capable of undergoing addition polymerization. Such free radically polymerizable compounds include mono-, di- or poly-(meth)acrylates (i.e., acrylates and methacrylates) such as, methyl (meth)acrylate, ethyl acrylate, isopropyl methacrylate, n-hexyl acrylate, stearyl acrylate, allyl acrylate, glycerol triacrylate, ethyleneglycol diacrylate, diethyleneglycol diacrylate, triethyleneglycol dimethacrylate, 1,3-propanediol di(meth)acrylate, trimethylolpropane triacrylate, 1,2,4-butanetriol trimethacrylate, 1,4-cyclohexanediol diacrylate, pentaerythritol tetra(meth)acrylate, sorbitol hexacrylate, tetrahydrofurfuryl (meth)acrylate, bis[1-(2-acryloxy)]-p-ethoxyphenyldimethylmethane, bis[1-(3-acryloxy-2-hydroxy)]-p-propoxyphenyldimethylmethane, ethoxylated bisphenolA di(meth)acrylate, and trishydroxyethyl-isocyanurate trimethacrylate; (meth)acrylamides (i.e., acrylamides and methacrylamides) such as (meth)acrylamide, methylene bis-(meth)acrylamide, and diacetone (meth)acrylamide; urethane (meth)acrylates; the bis-(meth)acrylates of polyethylene glycols (e.g., molecular weight 200-500), copolymerizable mixtures of acrylated monomers such as those in U.S. Pat. No. 4,652,274 (Boettcher et al.), acrylated oligomers such as those of U.S. Pat. No. 4,642,126 (Zador et al.), and poly(ethylenically unsaturated) carbamoyl isocyanurates such as those disclosed in U.S. Pat. No. 4,648,843 (Mitra); and vinyl compounds such as styrene, diallyl phthalate, divinyl succinate, divinyl adipate and divinyl phthalate. Other suitable free radically polymerizable compounds include siloxane-functional (meth)acrylates as disclosed, for example, in WO-00/38619 (Guggenberger et al.), WO-01/92271 (Weinmann et al.), WO-01/07444 (Guggenberger et al.), WO-00/42092 (Guggenberger et al.) and fluoropolymer-functional (meth)acrylates as disclosed, for example, in U.S. Pat. No. 5,076,844 (Fock et al.), U.S. Pat. No. 4,356,296 (Griffith et al.), EP-0373 384 (Wagenknecht et al.), EP-0201 031 (Reiners et al.), and EP-0201 778 (Reiners et al.). Mixtures of two or more free radically polymerizable compounds can be used if desired.

The polymerizable component may also contain hydroxyl groups and free radically active functional groups in a single molecule. Examples of such materials include hydroxyalkyl (meth)acrylates, such as 2-hydroxyethyl (meth)acrylate and 2-hydroxypropyl (meth)acrylate; glycerol mono- or di-(meth)acrylate; trimethylolpropane mono- or di-(meth)acrylate; pentaerythritol mono-, di-, and tri-(meth)acrylate; sorbitol mono-, di-, tri-, tetra-, or penta-(meth)acrylate; and 2,2-bis[4-(2-hydroxy-3-methacryloxypropoxy)phenyl]propane (bisGMA). Suitable ethylenically unsaturated compounds are also available from a wide variety of commercial sources, such as Sigma-Aldrich, St. Louis. Mixtures of ethylenically unsaturated compounds can be used if desired.

Particularly useful photopolymerizable components for use in the resin composition include PEGDMA (polyethyleneglycol dimethacrylate having a molecular weight of approximately 400), bisGMA, UDMA (urethane dimethacrylate), GDMA (glycerol dimethacrylate), TEGDMA (triethyleneglycol dimethacrylate), bisEMA6 as described in U.S. Pat. No. 6,030,606 (Holmes), and NPGDMA (neopentylglycol dimethacrylate). Various combinations of the polymerizable components can be used if desired.

For example, some embodiments of the resin composition can include approximately at least 10, 20, 30, 40, 50, 60, 70, 80, 85, 90, 92, 95, 96, 98 or 99% by weight of photopolymerizable components based on the total weight of the composition. In some embodiments, the resin composition can include approximately less than 10, 20, 30, 40, 50, 60, 70, 80, 85, 90, 92, 95, 96, 98 or 99% by weight of photopolymerizable components based on the total weight of the composition. In some embodiments, the resin composition can include a range, e.g., between approximately 10% by weight to approximately 99% by weight, between approximately 10% by weight to approximately 50% by weight, between approximately 50% by weight to approximately 99% by weight, or between approximately 40% by weight to approximately 70% by weight of photopolymerizable components based on the total weight of the composition.

In one embodiment, the resin composition, such as photopolymerizable components of one or more ethylenically unsaturated compounds, includes approximately at least 10, 15, 20, 25, 30, or 40% by weight based on the total weight of the resin composition. In some embodiments, the resin composition, such as photopolymerizable components of one or more ethylenically unsaturated compounds, includes approximately less than 60, 70, 75, 80, 85, or 90% by weight based on the total weight of the resin composition. In other embodiments, the resin composition, such as photopolymerizable components of one or more ethylenically unsaturated compounds, includes a range, e.g., between approximately 10% by weight to approximately 90% by weight, between approximately 10% by weight to approximately 40% by weight, between approximately 60% by weight to approximately 90% by weight, or between approximately 40% by weight to approximately 70% by weight based on the total weight of the resin composition.

In one embodiment, the resin composition, such as photopolymerizable components of one or more ethylenically unsaturated compounds with acid functionality and an initiator system, includes approximately at least 10, 15, 20, 25, 30, or 40% by weight based on the total weight of the resin composition. In some embodiments, the resin composition, such as photopolymerizable components of one or more ethylenically unsaturated compounds with acid functionality and an initiator system, includes approximately less than 60, 70, 75, 80, 85, or 90% by weight based on the total weight of the resin composition. In other embodiments, the resin composition, such as photopolymerizable components of one or more ethylenically unsaturated compounds with acid functionality and an initiator system, includes a range, e.g., between approximately 10% by weight to approximately 90% by weight, between approximately 10% by weight to approximately 40% by weight, between approximately 60% by weight to approximately 90% by weight, or between approximately 40% by weight to approximately 70% by weight based on the total weight of the resin composition.

Suitable photoinitiators (i.e., photoinitiator systems that include one or more compounds) for polymerizing free radically photopolymerizable resin compositions include binary and tertiary systems. Typical tertiary photoinitiators include an iodonium salt, a photosensitizer, and an electron donor compound as described in U.S. Pat. No. 5,545,676 (Palazzotto et al.). lodonium salts can include the diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Photosensitizers can include monoketones and diketones that absorb some light within a range of 400 nm to 520 nm, or 450 nm to 500 nm. Compounds can include alpha diketones that have some light absorption within a range of 400 nm to 520 nm or of 450 nm to 500 nm. Compounds can include camphorquinone, benzil, furil, 3,3,6,6-tetramethylcyclohexanedione, phenanthraquinone, 1-phenyl-1,2-propanedione and other 1-aryl-2-alkyl-1,2-ethanediones, and cyclic alpha diketones. Electron donor compounds can include substituted amines, e.g., ethyl dimethylaminobenzoate. Other suitable tertiary photoinitiator systems useful for photopolymerizing cationically polymerizable resins are described, for example, in U.S. Pat. Publication No. 2003/0166737 (Dede et al.).

Other suitable photoinitiators for polymerizing free radically photopolymerizable compositions include the class of phosphine oxides that typically have a functional wavelength range of 380 nm to 1200 nm. Phosphine oxide free radical initiators with a functional wavelength range of 380 nm to 450 nm can include acyl and bisacyl phosphine oxides such as those described in U.S. Pat. No. 4,298,738 (Lechtken et al.), U.S. Pat. No. 4,324,744 (Lechtken et al.), U.S. Pat. No. 4,385,109 (Lechtken et al.), U.S. Pat. No. 4,710,523 (Lechtken et al.), and U.S. Pat. No. 4,737,593 (Ellrich et al.), U.S. Pat. No. 6,251,963 (Kohler et al.); and EP Application No. 0 173 567 A2 (Ying).

In one embodiment, the resin composition includes an effective amount from approximately 0.1 wt % to approximately 5.0 wt % of one or more photoinitiators, based on the total weight of the resin composition.

The compositions can also contain fillers. Fillers may be selected from one or more of a wide variety of materials suitable for incorporation in compositions used for dental applications, such as fillers currently used in dental restorative compositions, and the like.

The filler can be finely divided. The filler can have a unimodial or polymodial (e.g., bimodal) particle size distribution. In some examples, the maximum particle size (the largest dimension of a particle, typically, the diameter) of the filler can be less than 20 micrometers, less than 10 micrometers, or less than 5 micrometers. In some examples, the average particle size of the filler can be less than 0.1 micrometers or less than 0.075 micrometer.

The filler can be an inorganic material. It can also be a crosslinked organic material that is insoluble in the resin system and is optionally filled with inorganic filler. The filler should in any event be nontoxic and suitable for use in the mouth. The filler can be radiopaque or radiolucent. The filler typically is substantially insoluble in water.

Examples of suitable inorganic fillers are naturally occurring or synthetic materials including, but not limited to: quartz; nitrides (e.g., silicon nitride); glasses derived from, for example, Zr, Sr, Ce, Sb, Sn, Ba, Zn, and Al; feldspar; borosilicate glass; kaolin; talc; titania; low Mohs hardness fillers such as those described in U.S. Pat. No. 4,695,251 (Randklev); and submicron silica particles (e.g., pyrogenic silicas such as those available under the trade designations AEROSIL, including “OX 50,” “130,” “150” and “200” silicas from Degussa Corp., Akron, Ohio and CAB-O-SIL M5 silica from Cabot Corp., Tuscola, Ill.). Examples of suitable organic filler particles include filled or unfilled pulverized polycarbonates, polyepoxides, and the like.

Non-acid-reactive filler particles can include quartz, submicron silica, and non-vitreous microparticles of the type described in U.S. Pat. No. 4,503,169 (Randklev). Mixtures of these non-acid-reactive fillers are also contemplated, as well as combination fillers made from organic and inorganic materials. In some embodiments, the filler can be silane-treated zirconia-silica (Zr—Si).

For some embodiments that include filler, the compositions can include at least 1% by weight, at least 2% by weight, and at least 5% by weight filler, based on the total weight of the composition.

In some embodiments, the resin composition includes less than about 1% by weight of optional additives such as, for example, preservatives (for example BHT), flavoring agents, indicators, dyes, pigments, inhibitors, accelerators, viscosity modifiers, wetting agents, buffering agents, radical and cationic stabilizers (for example BHT), and the like, based on the total weight of the resin composition.

In various embodiments, the film 124 can have a thickness no greater than needed to provide microstructures 122 that release therapeutic agents on a sustainable basis over a predetermined wear time. The film layer 124 forming the structured surface 110 should be sufficiently thin so not to substantially interfere with the dimensional tolerances or flexibility of the shell 102. In some embodiments, which are not intended to be limiting, the film layer 124 can have a thickness of less than 1 μm, but increased thicknesses may be used depending on the degree of therapeutic agent release needed over a period of time. In various embodiments, the film layer 124 has a thickness of about 1 nm to about 200 nm, or about 5 nm to about 85 nm, or about 10 nm to about 50 nm, or about 25 nm to about 40 nm.

In some embodiments, which are provided herein only as non-limiting examples, suitable thin film layers 124 can have a weight of approximately 0.007 g/cm². In other non-limiting examples, suitable thin film layers 124 can have a weight of approximately at least 0.1, 0.2, 0.5, 0.7, 1, 2, 3, or 6 mg/cm². In other non-limiting examples, suitable thin film layers 124 can have a weight of approximately less than 10, 20, 30, 40, 50 or even 70 mg/cm². In other non-limiting examples, suitable thin film layers 124 can have a weight of approximately 0.1 to approximately 6 mg/cm², approximately 10 to approximately 70 mg/cm², approximately 0.7 to approximately 30 mg/cm², approximately 0.5 to approximately 50 mg/cm², or approximately 6 to approximately 20 mg/cm².

In various embodiments, the microstructures 122 may be continuous or discontinuous over the structured surface 110. For example, referring again to FIG. 1, the structured surface 110 includes a region 118 that is substantially free of microstructures 122, a plurality of first regions 120 including a first arrangement of microstructures 122, a second region 130 including a dense array of microstructures 122, and a third region 140 having microstructures 122 of varying shapes or densities. For example, FIG. 2A shows the microstructures 122A in the first region 120, which are uniformly arranged and relatively large compared to the size of the microstructures 122B in the second region as shown in FIG. 2B. The third region 140 in FIG. 2C includes 3D microstructures 122C that are non-uniformly arranged and have varying shapes and sizes.

In various embodiments, one region of microstructures 122 can be spread continuously over the entire surface of the polymeric shell 102 or a selected portion of the surface of the polymeric shell 102. In some embodiments, two or more regions of microstructures 122 can be varied uniformly or randomly on at least a portion of the polymeric shell 102. For example, a first region of microstructures 122 with a first shape or size can be disposed on the front portion 112 of the first major external surface 106 of the polymeric shell 102, and a second region of microstructures 122 with a different shape or size can be disposed only on the sides 114A and 114B of the second major internal surface 108 of the polymeric shell 102.

In various embodiments, the microstructures 122 of the structured surface 110 can have varying cross-sectional shapes including, but not limited to, triangular, circular, lenticular, elliptical, conical, irregular, and combinations thereof. The cross-sectional shapes of the microstructures 122 in a first region of the structured surface 110 can be the same or different from the cross-sectional shapes of microstructures in other regions of the structured surface 110.

The microstructures 122 can be uniformly arranged or randomly distributed on the structured surface 110 on the polymeric shell 102, and randomly distributed on other portions of the polymeric shell 102. For example, the microstructures 122 can be arranged uniformly on the first major external surface 106 and distributed randomly on the second major internal surface 108 of the polymeric shell 102. In other examples, the microstructures 122 can be arranged uniformly on the sides 114A and 114B and distributed randomly on the front 112 of the second major internal surface 108 of the structured surface 110 on the shell 102.

In various embodiments, the height, cross-sectional width, or both, of the three-dimensional microstructures 122 above the structured surface 110 can vary between regions on the surfaces 106, 108 of the polymeric shell 102, and even can vary within a selected region.

In some embodiments, the microstructures 122 can be a plurality of engineered microstructures on at least a portion of the structured surface, wherein each microstructure of the plurality of microstructures comprises a base having at least one microscale cross-sectional dimension. The aspect ratio of each microstructure can be approximately at least about 0.5, no greater than about 10, or a range between about 0.5 and about 10, e.g., a range between about 1 and about 3.

As used herein, a “microstructure” can be a structure or feature having a recognizable geometric shape defined by a volume that projects out of the base plane of a surface or an indented volume which projects into the surface. Such structures can include a base having cross sectional dimensions no less than about 50 μm, no greater than about 1000 μm, a range between about 50 μm and about 1000 μm, e.g., a range between about 200 μm and about 300 μm, or about 250 μm. As used herein, an “engineered microstructure” can mean a microstructure deliberately formed into and integral with a surface. An engineered microstructure may be created, for example, by microreplicating a specific pattern unto a surface. An engineered microstructure is distinct from structures produced by random application of particles, by spraying, painting, dipping, adhesive bonding, etc., to a surface.

In some embodiments, which are provided as a non-limiting example, the height of the microstructures 122 can vary from of about 25 μm to about 750 μm as measured from a plane of the structured surface 110. In some embodiments, the height of the microstructures 122 can be as least about 50 μm, at least about 100 μm, or at least about 200 μm. In the same or other embodiments, the height of the microstructures can be no greater than about 600 μn, no greater than about 500 μm no greater than 300 μm or no greater than about 200 μm. Typical ranges for microstructure 122 height include from about 50 μm to about 300 μm, or about 100 μm to about 200 μm. In some embodiments, the height of the microstructures 122 is substantially the same, but some variation in height can be tolerated while maintaining good performance. For example, in some embodiments, the average height of the microstructures 122 can vary by ±50 μm, while in other embodiments the average height can vary by ±10 μm, and in yet other embodiments the average height can vary by ±1 μm, while maintaining acceptable performance.

In some embodiments, which are provided as an example, the microstructures 122 have an average density of about 100 per cm² up to about 6000 per cm² on the structured surface 110, or about 100 per cm² to about 3500 per cm², or about 200 per cm² to about 2500 per cm².

In some exemplary embodiments, the microstructures 122 have an average period (i.e., the center to center distance between adjacent microstructures) of about 50 μm to about 2000 μm, or about 200 μm to about 1000 μm. Although the average period of the microstructures 122 can be substantially the same, such an arrangement is not required. In some examples, the average period between adjacent microstructures of the plurality of engineered microstructures can be at least 1 time and no greater than 5 times than the smallest cross-sectional dimension, such as the cross-sectional width of the base.

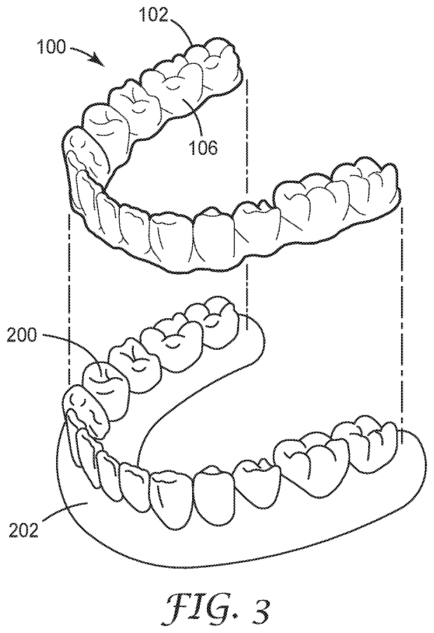

Referring now to FIG. 3, the shell 102 of the orthodontic appliance 100 is an elastic polymeric material that generally conforms to a patient's teeth 200, but that is slightly out of alignment with the patient's initial tooth configuration. In some embodiments, the shell 102 may be one of a group or a series of shells having substantially the same shape or mold, but which are formed from different materials to provide a different stiffness or resilience as need to move the teeth of the patient. In this manner, in one embodiment, a patient or a user may alternately use one of the orthodontic appliances during each treatment stage depending upon the patient's desired usage time or treatment time period for each treatment stage.

No wires or other means may be provided for holding the shell 102 over the teeth 200, but in some embodiments, it may be desirable or necessary to provide individual anchors on teeth with corresponding receptacles or apertures in the shell 102 so that the shell 102 can apply a retentive or other directional orthodontic force on the tooth which would not be possible in the absence of such an anchor.

The shells 102 may be customized, for example, for day time use and night time use, during function or non-function (chewing vs. non-chewing), during social settings (where appearance may be more important) and nonsocial settings (where the aesthetic appearance may not be a significant factor), or based on the patient's desire to accelerate the teeth movement (by optionally using the more stiff appliance for a longer period of time as opposed to the less stiff appliance for each treatment stage).

For example, in one aspect, the patient may be provided with a clear orthodontic appliance that may be primarily used to retain the position of the teeth, and an opaque orthodontic appliance that may be primarily used to move the teeth for each treatment stage. Accordingly, during the day time, in social settings, or otherwise in an environment where the patient is more acutely aware of the physical appearance, the patient may use the clear appliance. Moreover, during the evening or night time, in non-social settings, or otherwise when in an environment where physical appearance is less important, the patient may use the opaque appliance that is configured to apply a different amount of force or otherwise has a stiffer configuration to accelerate the teeth movement during each treatment stage. This approach may be repeated so that each of the pair of appliances are alternately used during each treatment stage.

Referring to FIG. 3, systems and method in accordance with the various embodiments include a plurality of incremental position adjustment appliances, each formed from the same or a different material, for each treatment stage of orthodontic treatment. The orthodontic appliances may be configured to incrementally reposition individual teeth 200 in an upper or lower jaw 202 of a patient. In some embodiments, the cavities 104 are configured such that selected teeth will be repositioned, while others of the teeth will be designated as a base or anchor region for holding the repositioning appliance in place as it applies the resilient repositioning force against the tooth or teeth intended to be repositioned.

Placement of the elastic positioner shell 102 over the teeth 200 applies controlled forces in specific locations to gradually move the teeth into the new configuration. Repetition of this process with successive appliances having different configurations eventually moves a patient's teeth through a series of intermediate configurations to a final desired configuration.

As noted above, in some embodiments the microstructures 122 on the structured surface 110 can be formed using casting-and-curing, microreplication, and combinations thereof, and then laminated to a substantially flat sheet of polymeric material, or a layer of adhesive may optionally be used to bond the components. The major surface of the polymeric sheet to which the structured surface 110 is applied may optionally be chemically or mechanically treated prior to applying the structured surface 110 to, for example, enhance adhesion between the structured surface 110 and the shell 102.

A plurality of cavities 104 may then be formed in the sheet of polymeric material to form the orthodontic appliance 100, wherein the cavities are configured to receive one or more teeth. The cavities 104 may be formed by any suitable technique, including thermoforming, laser processing, chemical or physical etching, and combinations thereof.

In another embodiment, the tooth-shaped 104 cavities may be formed in the sheet of polymeric material to form a shell-like orthodontic dental appliance 100, and then microstructures 122 may thereafter be formed on or applied to overlie all or a desired portion of the cavities 104. In some embodiments, the structured surface 110 may also be formed on or applied on all or a desired portion of an external surface 106 of the dental appliance 100 opposite the teeth-retaining cavities 104.

In another embodiment, the shell-like orthodontic dental appliance 100 may be formed using a three-dimensional (3D) printing process (e.g., additive manufacturing), such as stereolithography, Fused Deposition Modeling, stereolithography (SLA), inverse vat polymerization, inkjet/polyjet printing, or other methods of additive manufacturing. In some embodiments, the microstructures 122 can be printed on at least one surface 106, 108 of the polymeric shell 102 during the 3D printing process, or the structured surface 110 may be applied on the surfaces 106, 108 following 3D printing or thermoforming. In some embodiments, the microstructures 122 may be made from the same material as the shell 102. In some examples, shell 102 may include a unitary material, e.g., a single, uniform material. The unitary material may include a single polymer, or homogeneous mixture of one or more polymers. For example, removable dental appliance 100 may consist of a single, continuous 3D printed or thermoformed component. In other examples, shell 102 may include a multi-layer material. Multi-layer materials may enable one or more portions of shell 102 to be formed with a plurality of layers having different elastic modulus to enable selection of force characteristics, displacement characteristics, or both. The multi-layer material may include multiple layers of a single material, e.g., a single polymer, or multiple layers of a plurality of materials, e.g., two or more polymers, a polymer and another material. Suitable polymers may include, but are not limited to, (meth)acrylate polymer; epoxy; silicones; polyesters; polyurethanes; polycarbonate; thiol-ene polymers; acrylate polymers such as urethane (meth)acrylate polymers, polyalkylene oxide di(meth)acrylate, alkane diol di(meth)acrylate, aliphatic (meth)acrylates, silicone (meth)acrylate; polyethylene terephthalate based polymers such as polyethylene terephthalate glycol (PETG); polypropylene; ethylene-vinyl acetate; or the like.

For example, orthodontic dental appliance 100 may consist of a multilayer or multi-material, 3D printed (i.e., created by additive manufacturing) component. Suitable 3D printing techniques include stereolithography (SLA), inverse vat polymerization, inkjet/polyjet printing, fused deposition modeling, or other methods of additive manufacturing. In certain embodiments, the microstructures 122 on one surface of the shell may contain different materials or material properties than the shell 102 itself or other microstructures on the same or different surfaces. In examples in which the shell and/or microstructures are created by, for example, polyjet printing, the printer may be configured to print multiple materials in a single print, thereby allowing a first material for the certain components of dental appliance (e.g., shell 102) and a second material for the microstructures 122. Further, with polyj et additive manufacturing, the modulus may be varied selectively across the shell 102, and a different modulus may be used for the microstructures than is used for the shell, for different regions of the microstructures (e.g., interior vs. exterior surfaces), or for different parts of the shell, for example.

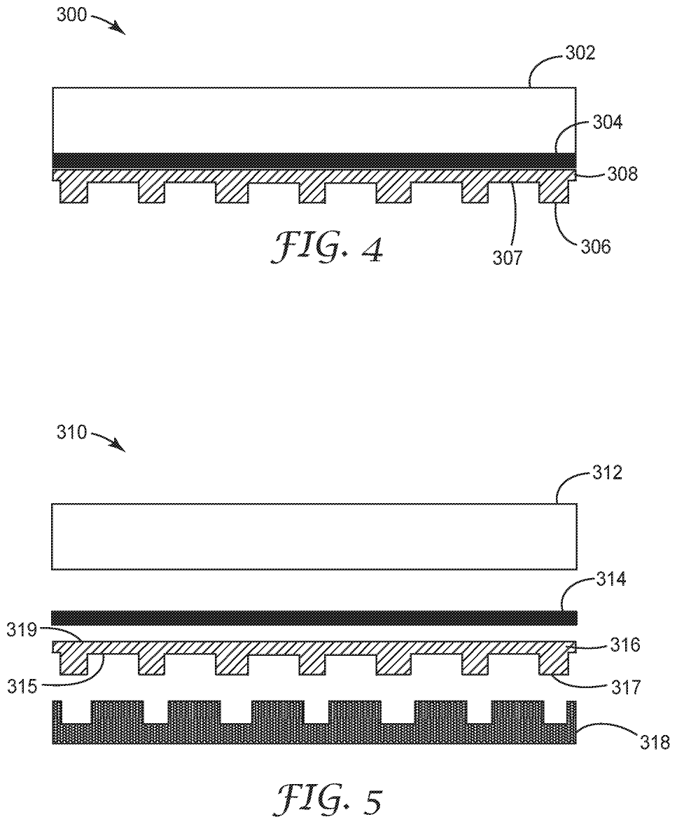

FIG. 4 shows an example of a dental appliance component 300 including a thermoplastic polymeric substrate 302 coated with an optional primer-adhesive layer 304. A structured layer 308 resides on the primer-adhesive layer 304 and includes a surface 307 including three dimensional (3D) microstructures 306 extending outward therefrom. In some examples, suitable primer-adhesive layer 304 can include components chosen from silanes, ethylenically unsaturated silanes, ethylenically unsaturated compounds, ethylenically unsaturated compounds with acid functionality, solvents, initiator systems, and wetting agents. The component 300 can subsequently be treated to include a suitable arrangement of teeth-retaining cavities by suitable methods including, for example, thermoforming, laser ablation, and the like.

FIG. 5 shows a cross-sectional exploded view of a subassembly 310 that can be used to form the dental appliance component of FIG. 4. The subassembly 310 includes a thermoplastic substrate 312 and an optional primer-adhesive layer 314. A hardenable resin composition can be cast on a suitable microstructured tool 318 and then separated from the microstructured tool 318 to form a microstructured film layer 316 with microstructures 317 extending outward from a first major surface 315 thereof. In some embodiments, the hardenable resin composition is photocurable, and can be hardened by any suitable technique including, but not limited to, chemical curing, photocuring, UV curing or electron-beam curing.

In some embodiments, the microstructured tool 318 of the subassembly 310 is an elastic polymeric material that may be transparent, translucent, or opaque. In some embodiments, the tool 318 is a clear or substantially transparent polymeric material that may include, for example, one or more of amorphous thermoplastic polymers, semi-crystalline thermoplastic polymers and transparent thermoplastic polymers chosen from polycarbonate, thermoplastic polyurethane, acrylic, polysulfone, polypropylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone and combinations thereof. In another embodiment, the tool 318 may be chosen from clear or substantially transparent semi-crystalline thermoplastic, crystalline thermoplastics and composites, such as polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof. In some embodiments, the tool 318 is a polymeric material chosen from polyethylene terephthalate, polyethylene terephthalate glycol, polycyclohexylenedimethylene terephthalate glycol, and mixtures and combinations thereof. One example of a commercially available material suitable as the elastic polymeric material for the tool 318, which is not intended to be limiting, is PETg. Suitable PETg resins can be obtained from various commercial suppliers such as, for example, Eastman Chemical, Kingsport, Tenn.; SK Chemicals, Irvine, Calif.; DowDuPont, Midland, Mich.; Pacur, Oshkosh, Wis.; and Scheu Dental Tech, Iserlohn, Germany.

In some embodiments, the tool 318 may be made of a single polymeric material or may include multiple layers of different polymeric materials. In some embodiments, the tool 318 may be made of a metallic surface. The tool 318 can be in the form of a flat sheet or steel roll where in the female image of the engineered microstructure can be inscribed via photolithographic or laser etching, diamond turning, etc. In some embodiments, the tool 318 can be made of copper, nickel, stainless steel and a combination thereof.

A second major surface 319 of the microstructured film layer 316 can be attached to the primer-adhesive layer 314, or directly on the substrate 312.

The resin hardening step on the microstructured film tool 318 can be completed prior to or after the second major surface 319 of the microstructured film layer 316 is attached to the primer-adhesive layer 314, or to the substrate 312. In some embodiments, the microstructured film tool 318 can be removed from the microstructured film layer 316 by simply peeling the film tool 318 away from the film layer 316 to expose the microstructures 317. In various embodiments, which are not intended to be limiting, the microstructured tool may include a microstructured polymeric film, a metal foil, and the like.

Embodiments will now be illustrated with reference to the following non-limiting examples.

### EXAMPLES

Referring again to FIG. 5, the thermoplastic substrate 312 was an optically-clear 0.75 mm thick PETg film, 12.5 cm diameter available from Scheu Dental Tech, Iserlohn, Germany under the trade designation DURAN.

The primer-adhesive 314 was a dental adhesive available from 3M Co, St. Paul, Minn., USA under the trade designation 3M SCOTCHBOND UNIVERSAL ADHESIVE (SBU).

The resin composition 316 for casting and hardening was a dental sealant available from 3M Co., St. Paul, Minn., USA, under the trade designation 3M CLINPRO SEALANT. 3M CLINPRO SEALANT is a photocurable TEGDMA based resin with therapeutic fluoride compound.

The cast film tool 318 was a polymeric (e.g., polypropylene) microreplication film for molding the liquid resin 316. The cavities in the cast film tool 318 included cylindrical holes that were approximately 300 μm in diameter and approximately 150 μm deep, approximately 4500 holes/inch, and approximately 350 mm center-to-center spacing between each hole.

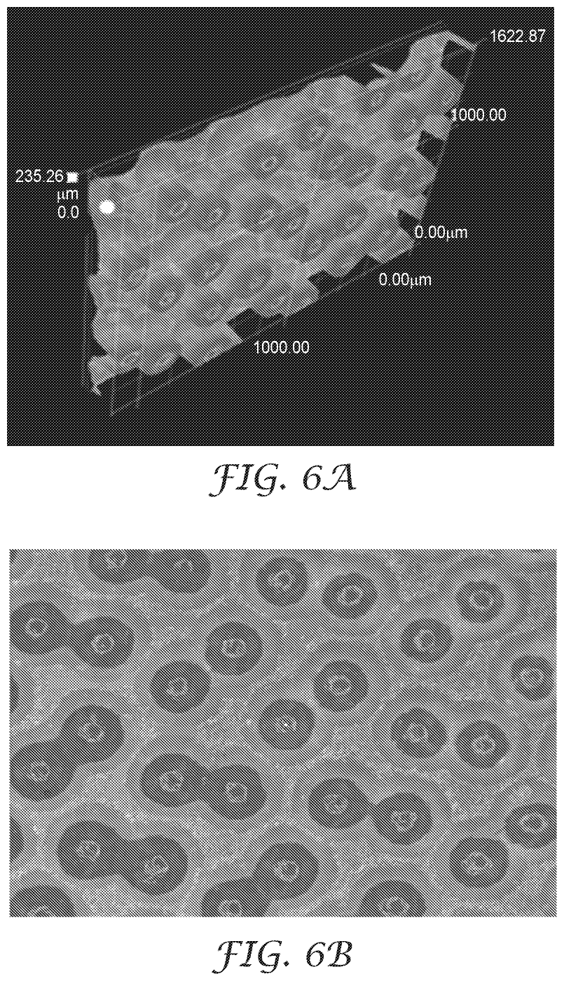

The cross-section and top views of the cast film tool 318 as obtained with a Keyence VHX Digital Microscope available from Keyence Corporation, Osaka, Japan, are shown in FIGS. 6A and 6B.

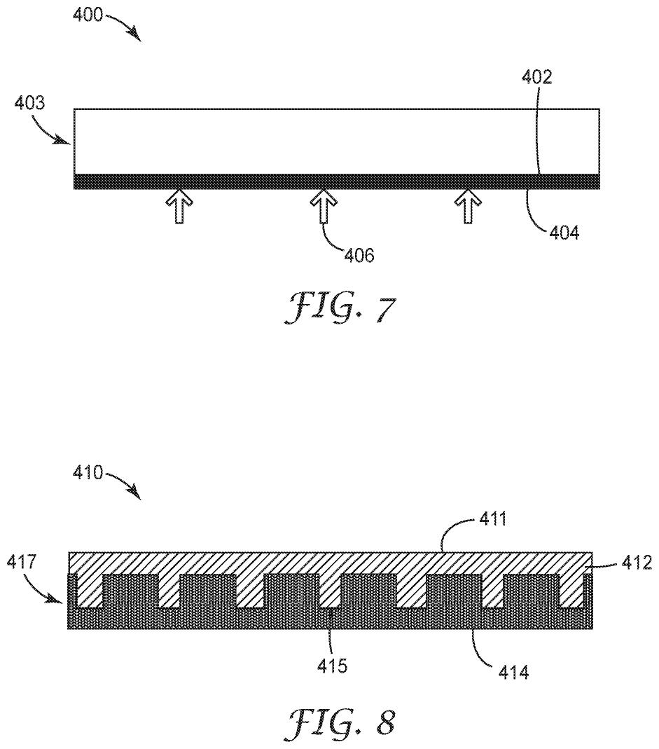

Referring to the schematic diagram of FIG. 7, in a first step 400 a thin uniform coating of SBU adhesive primer 404 was applied to one surface of a 12.5 cm diameter DURAN PETg disc 402 with a squeeze. The primer layer 404 was gently dried with oil-free compressed air for 20 seconds, and then crosslinked for 2 minutes with visible blue light emitted by a LED light available from 3M under the trade designation S10 ELIPAR LED light to form a first component 403.

Referring now to FIG. 8, in a second step 410 a cast film tool material 414 as described above was cut into a 12.5 cm square. Under gold room lights, to filter out blue wavelengths, the resin sealant 412 (3M CLINPRO SEALANT) was gently applied on the entire surface of the tool with a squeeze to fill all the cylindrical cavities 415 without air entrapment. To remove all entrapped air from the resin 412 and the tool cavities 415, the coated tool 414 was vacuumed in a desiccator for 10 min. After ensuring that all the cavities 415 were bubble free, resin 412 was added or removed as necessary to cover the landing space between the cavities (approximately 1-mil thick) to form a second component 417 with an exposed major surface 411.

Referring now to FIG. 9, in a third step 420 the major surface 411 of the resin-filled second component 417 (FIG. 8) was laminated to the adhesive layer 404 of the first component (FIG. 7) with no entrapped air between the surfaces. The resulting sandwich construction 430 was placed in the center of two glass plates (7-in×5-in×0.5-in) (not shown in FIG. 9). The top glass plate was loaded with a 2 kg weight (not shown in FIG. 9, downward pressure shown schematically by arrows 434) to ensure good interlayer contact within the sandwich construction 430. The layers 404, 412 were then polymerized with visible blue light 432 emitted by the S10 Elipar LED light for 4 min exposure time. The sandwich construction 430 was then removed from between the glass plates and the layers 404, 412 were further polymerized for an additional 4 min with UV-Vis LED lights available under the trade designation CF2000 from Clearstone Technologies, Hopkins, Minn., USA.

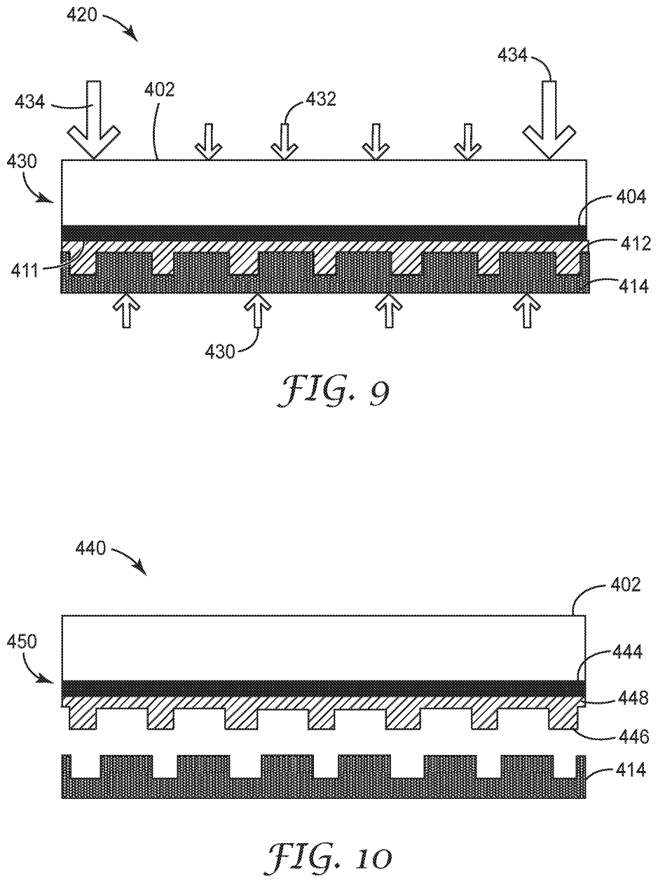

In a final step 440 shown schematically in FIG. 10, the casting tool 414 was removed from the hardened resin layer 448 to expose microstructures 446 and produce an article 450 in which the resin layer 448 was attached to the substrate 402 via the hardened primer adhesive layer 444.

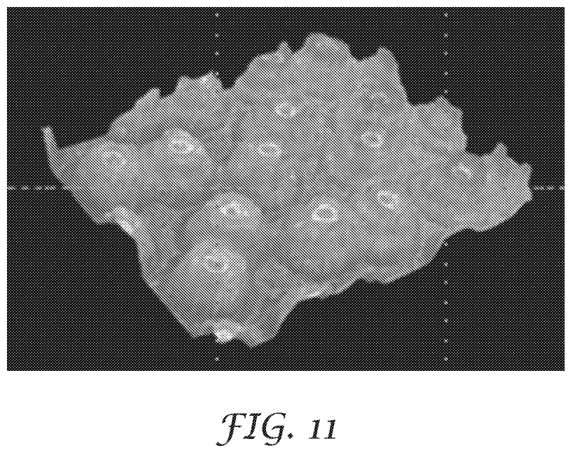

A topographical view of the 3D microstructured coating on a portion of the article 450 of FIG. 10 is shown in FIG. 11.

In one embodiment, the article 450 of FIG. 10 was then thermoformed into an aligner tray having microstructures on its inside surface intended to be placed adjacent to the teeth of a patient.

In another embodiment, the article 450 of FIG. 10 was thermoformed into an aligner tray before separating the tool 414 from the article, exposing the microstructures on its inside surface intended to be placed adjacent to the teeth of a patient. In some examples, a polymer surface of the article 450 and a polymer surface of the tool 414 are thermoformed together into a laminate construction and then the polymer surface of the tool 414 is removed from the article 450, e.g., a dental appliance, to expose the 3-D microstructure.

For fluoride release testing, fluoride release was measured on a T70 titrator (available from Mettler Toledo, Columbus, Ohio, USA). The fluoride electrode (available from Cole-Parmer, Vernon Hills, Ill., USA) was first calibrated with parts per million (ppm) fluoride standards prepared from Total Ionic Strength Adjustment Buffer (TISAB II) before measuring samples for fluoride release each day. The fluoride release was calculated against the fluoride standards calibration curve.

### Example 1

Using the process described above, the 3M CLINPRO SEALANT was used to generate a hardened microtextured surface including cylindrical posts having a diameter of approximately 100 μm, a height of approximately 150 μm, and a post spacing of approximately 250 μm on a 30 mil PETg substrate as shown in FIG. 11. The weight of the microtextured coating was 0.007 g/cm². A total of five PETg disks (2 cm diameter) were made per coating and each disk was stored in a plastic centrifuge tube (50 mL) with 25 mL of MilliQ water at 37° C. The centrifuge tubes were aged with their contents upright in an oven at 37° C. The experiment was carried out over a period of one week with data collection points after 1 day, 3 days, and 7 days. At each time point, a 5 mL aliquot from each centrifuge tube was mixed with 5 mL TISAB II to measure F-ion (parts per million) content using a calibrated ion-selective electrode.

Table 1 shows the difference in fluoride ion release between a substrate with a microstructured fluoride releasing coating versus a control substrate with no coating over the time period between zero and seven days.

TABLE 1
Fluoride Ion Release from a Cast
and Cure Micro-Structured Surface

| Test Time | F⁻ ion release ppm | F⁻ ion release ppm |
|---|---|---|
| Interval | Control | Coated Example 1 |
| Day 0 | 0.0 | 0.0 |
| Day 1 | 0.04 | 0.19 |
| Day 3 | 0.06 | 0.28 |
| Day 7 | 0.09 | 0.33 |

Various embodiments of the invention have been described. These and other embodiments are within the scope of the following claims.

### Embodiments

1. A dental appliance, comprising:

a polymeric substrate comprising a plurality of cavities for receiving one or more teeth; and

an arrangement of engineered microstructures on the substrate, wherein the engineered microstructures comprise a therapeutic agent.
2. The dental appliance of embodiment 1, wherein the arrangement of engineered microstructures comprises an array of three-dimensional engineered microstructures extending upward from a surface of the polymeric substrate.
3. The dental appliance of embodiment 2, wherein the three-dimensional engineered microstructures have a cross-sectional shape chosen from triangular, circular, lenticular, elliptical, conical, and combinations thereof.
4. The dental appliance of embodiments 2-3, wherein the three-dimensional engineered microstructures are continuous over a region of the surface of the polymeric substrate.
5. The dental appliance of embodiment 2-3, wherein the three-dimensional engineered microstructures are randomly distributed on the surface of the polymeric substrate.
6. The dental appliance of embodiments 2-5, wherein the therapeutic agent comprises a fluoride compound, a calcium compound, a phosphate compound, or a combination thereof.
7. The dental appliance of embodiment 6, wherein the fluoride compound is releasable from the three-dimensional engineered microstructures over a predetermined patient wear time of the dental appliance.
8. The dental appliance of embodiments 2-7, wherein the polymeric substrate comprises a thermoplastic polymer chosen from polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.
9. The dental appliance of embodiment 8, wherein the polymeric substrate is a polymeric material chosen from polyethylene terephthalate, polyethylene terephthalate glycol, poly cyclohexylenedimethylene terephthalate glycol, and mixtures and combinations thereof.
10. The dental appliance of embodiments 1-9, wherein the dental appliance transmits at least 60% of incident light with a wavelength of between about 400 nanometers (nm) to about 750 nm.
11. The dental appliance of embodiment 10, wherein the dental appliance transmits at least 90% of incident light with a wavelength of between about 400 nanometers (nm) to about 750 nm.
12. A dental appliance, comprising:

a polymeric substrate comprising a plurality of cavities for receiving one or more teeth; and

a polymeric film on a major surface of the polymeric substrate, wherein a surface of the polymeric film comprises an array of three-dimensional engineered microstructures extending upward from the surface, and wherein the three-dimensional engineered microstructures comprise a compound releasable from the three-dimensional engineered microstructures over a predetermined patient wear time of the dental appliance.
13. The dental appliance of embodiment 12, further comprising an adhesive layer between the polymeric substrate and the polymeric film.
14. The dental appliance of embodiment 13, wherein the adhesive layer extends substantially continuously on the major surface the substrate.
15. The dental appliance of embodiments 13-14, wherein the adhesive has a thickness of between about 1 nanometer to about 25 micrometers.
16. The dental appliance of embodiments 12-15, wherein the polymeric film has a thickness of between about 50 micrometers (μm) to about 3,000 μm.
17. The dental appliance of embodiments 12-16, wherein the substrate comprises a thermoplastic polymer is chosen from polyamide, polyethylene terephthalate, polybutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.
18. The dental appliance of embodiments 12-17, wherein the surface of the polymeric film comprises at least a first region and a second region, and wherein the array of three-dimensional engineered microstructures in the first region is different than the array in the second region.
19. The dental appliance of embodiment 18, wherein the cross-sectional shape of the three-dimensional engineered microstructures in the first region differs from the cross-sectional shape of the three-dimensional engineered microstructures in the second region.
20. The dental appliance of embodiment 18 or 19, wherein the height of the three-dimensional engineered microstructures in the first region is different from the height of the three-dimensional engineered microstructures in the second region.
21. The dental appliance of embodiment 18, wherein the height of the three-dimensional engineered microstructures in the second region varies.
22. The dental appliance of embodiment 18, 19, 20 or 21, wherein the cross-sectional width of the three-dimensional engineered microstructures of the first region differs from the cross-sectional width of the three-dimensional engineered microstructures in the second region.
23. The dental appliance of embodiment 18, 19, 20, 21, or 22, wherein the therapeutic agents released from the three-dimensional engineered microstructures in the first region are different from the therapeutic agents released from the three-dimensional engineered microstructures in the second region.
24. The dental appliance of embodiment 18, 19, 20, 21, 22 or 23, wherein the therapeutic agents of the three-dimensional engineered microstructures in the first region are released at a different concentration than the therapeutic agents of the three-dimensional engineered microstructures in the second region.
25. The dental appliance of embodiments 12-24, wherein the three-dimensional engineered microstructures are continuous on the outwardly facing surface of the polymeric film.
26. The dental appliance of embodiments 12-24, wherein the three-dimensional engineered microstructures are discontinuous on the outwardly facing surface of the polymeric film.
27. The dental appliance of embodiments 12-24, wherein the three-dimensional engineered microstructures are distributed randomly on the outwardly facing surface of the polymeric film.
28. The dental appliance of embodiments 12-27, wherein the cross-sectional shape of the three-dimensional engineered microstructures is chosen from triangular, circular, lenticular, elliptical, conical, and combinations thereof.
29. The dental appliance of embodiments 12-28, wherein the dental appliance transmits at least 90% of incident light with a wavelength of between about 400 nanometers (nm) to about 750 nm. 30. A method of making a dental appliance, the method comprising:

attaching to a first major surface of a first polymeric film to a first major surface of a second polymeric film to form a laminate construction, wherein a second major surface of the second polymeric film comprises an array of three-dimensional engineered microstructures extending outward therefrom, and wherein the three-dimensional engineered microstructures comprise a compound releasable from the three-dimensional engineered microstructures; and

shaping the laminate construction to comprise an arrangement of cavities configured to receive one or more teeth.
31. The method of embodiment 30, comprising forming the array of three-dimensional engineered microstructures on the second polymeric film by casting a polymeric resin on a surface of a tool.
32. The method of embodiments 30-31, wherein shaping the laminate construction comprises thermoforming the laminate construction and removing the second polymeric film from a tool to form in the laminate construction the arrangement of cavities.
33. The method of embodiments 30-32, wherein the first polymeric film comprises a thermoplastic polymer chosen from polyamide, polyethylene terephthalate, polybutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.
34. The method of embodiments 30-33, wherein the second polymeric film comprises a thermoplastic polymer chosen from polyamide, polyethylene terephthalate, polybutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.
35. The method of embodiments 30-34, wherein the first major surface of the first polymeric film comprises an adhesive layer.
36. The method of embodiment 35, wherein the adhesive layer comprises a primer, a bonding agent and combinations thereof.
37. The method of embodiments 30-36, wherein the three-dimensional engineered microstructures have a cross-sectional shape chosen from triangular, circular, lenticular, elliptical, conical, and combinations thereof.
38. The method of embodiments 30-37, wherein the three-dimensional engineered microstructures are continuous over a region of the second major surface of the second polymeric film.
39. The method of embodiments 30-37, wherein the three-dimensional engineered microstructures are randomly distributed on the second major surface of the second polymeric film.
40. The method of embodiments 30-39, wherein the dental appliance transmits at least 60% of incident light with a wavelength of between about 400 nanometers (nm) to about 750 nm.
41. The method of embodiment 40, wherein the dental appliance transmits at least 90% of incident light with a wavelength of between about 400 nanometers (nm) to about 750 nm.
42. A method of dental treatment, comprising:

positioning a dental appliance around one or more teeth, wherein the dental appliance comprises a plurality of cavities for receiving one or more teeth, and an array of three-dimensional engineered microstructures on an exposed surface of the dental appliance, wherein the engineered microstructures comprise one or more therapeutic agents; and

releasing the therapeutic agents into the mouth of a patient.
43. The method of embodiment 42, wherein the therapeutic agents comprise fluoride.
44. The method of embodiment 41 or 42, wherein the therapeutic agents are gradually released onto the teeth of the patient over a predetermined release time period.

1-7. (canceled)

8. A dental appliance, comprising:
a polymeric substrate comprising a plurality of cavities for receiving one or more teeth; and
a polymeric film on a major surface of the polymeric substrate, wherein a surface of the polymeric film comprises an array of three-dimensional engineered microstructures extending outward from the surface, and wherein the three-dimensional engineered microstructures comprise a compound releasable from the three-dimensional engineered microstructures over a predetermined patient wear time of the dental appliance.

9. The dental appliance of claim 8, further comprising an adhesive layer between the polymeric substrate and the polymeric film.

10. The dental appliance of claim 9, wherein the adhesive layer extends substantially continuously on the major surface the substrate.

11. The dental appliance of claim 8, wherein the substrate comprises a thermoplastic polymer chosen from polyamide, polyethylene terephthalate, polybutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.

12. The dental appliance of claim 11, wherein substrate comprises a single layer material or multi-layer material, wherein the single layer material includes a single material or a plurality of materials, and wherein the multi-layer material includes multiple layers of a single material, or multiple layers of a plurality of materials.

13. The dental appliance of claim 8, wherein the surface of the polymeric film comprises at least a first region and a second region, and wherein the array of three-dimensional engineered microstructures in the first region is different than the array in the second region.

14. The dental appliance of claim 13, wherein at least one of the cross-sectional shape, height, and cross-section width of the three-dimensional engineered microstructures in the first region differs from the corresponding cross-sectional shape, height, or cross-sectional width of the three-dimensional engineered microstructures in the second region.

15. The dental appliance of claim 13, wherein the height of the three-dimensional engineered microstructures in the second region varies.

16. The dental appliance of claim 13, wherein the therapeutic agents released from the three-dimensional engineered microstructures in the first region are at least one of different from the therapeutic agents released from the three-dimensional engineered microstructures in the second region, and released at a different concentration than the therapeutic agents of the three-dimensional engineered microstructures in the second region.

17. A method of making a dental appliance, the method comprising:
attaching to a first major surface of a first polymeric film to a first major surface of a second polymeric film to form a laminate construction, wherein a second major surface of the second polymeric film comprises an array of three-dimensional engineered microstructures extending outward therefrom, and wherein the three-dimensional engineered microstructures comprise a compound releasable from the three-dimensional engineered microstructures; and
shaping the laminate construction to comprise an arrangement of cavities configured to receive one or more teeth.

18. The method of claim 17, comprising forming the array of three-dimensional engineered microstructures on the second polymeric film by casting a polymeric resin on a surface of a tool.

19. The method of claim 17, wherein shaping the laminate construction comprises thermoforming the laminate construction and removing the second polymeric film from a tool to form in the laminate construction the arrangement of cavities.

20. The method of claim 17, wherein the first polymeric film comprises a thermoplastic polymer chosen from polyamide, polyethylene terephthalate, polybutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof, and wherein the second polymeric film comprises a thermoplastic polymer chosen from polyamide, polyethylene terephthalate, polydbutylene terephthalate, polyester, polyester/polycarbonate copolymer, polyolefin, cyclic olefin polymer, styrenic copolymer, polyetherimide, polyetheretherketone, polyethersulfone, polytrimethylene terephthalate, and mixtures and combinations thereof.

21. The method of claim 17, wherein the first major surface of the first polymeric film comprises an adhesive layer comprising a primer, a bonding agent and combinations thereof.
